(19) United States
(12) Patent Application Publication
(10) Pub. No.: US 2014/0148745 A1
(43) Pub. Date: May 29, 2014

(54) ADJUSTABLE CUFF KNEE BRACE

(71) Applicant: Asterisk. Astrisk LLC, Lake Forest, CA (US)

(72) Inventor: Daniel Castillo, Mission Viejo, CA (US)

(21) Appl. No.: 13/688,965

(22) Filed: Nov. 29, 2012

(51) Int. Cl. A61F 5/01

(52) U.S. Cl. CPC A61F 5/0102
USPC 602/16

(57) ABSTRACT

Provided is a knee brace configured to be easily adjustable to conform to the size of the wearer's limb. Along these lines, the knee brace includes at least one adjustable cuff and an adjustment member coupled to the adjustable cuff for effectuating adjustment of the cuff. The adjustment member is exteriorly positioned relative to the cuff to allow the adjustment member to apply a compressing force upon the cuff to tighten the cuff on the wearer's limb.

### CROSS-REFERENCE TO RELATED APPLICATIONS

Not Applicable.

### STATEMENT RE: FEDERALLY SPONSORED RESEARCH/DEVELOPMENT

Not Applicable

### BACKGROUND OF THE INVENTION

1. Technical Field of the Invention

The present invention relates generally to braces for human joint support, and more specifically to an exteriorly positionable anatomical brace having adjustable cuffs coupled to respective limb structures on either side of a pivoting joint, such as a knee joint, to thereby provide adjustable tightening pressure on the limb structures resulting in stability to the supported joint.

2. Description of the Related Art

Injury and disease can affect the health, well-being and operability of various joints of the human body. The knee and elbow are among the most important joints in the body, and are susceptible to disease, such as osteo-arthritis, which can curtail normal activity, or injury, such as a sports related abuse or impact, which can prevent or severely limit continued activity.

One manner of treating such joint conditions is to fit the wearer with an appropriate brace whereby a pivotal support member is positioned adjacent the affected joint. When worn properly, a suitable brace may stabilize and support the pivoting joint through pivotal movement to prevent the joint from extending beyond an allowable pivotal range of motion. Such support may protect the wearer from joint injuries, including ligament and cartilage injuries.

Proper usage of the brace typically requires that the brace is properly fitted to the user and securely held in place adjacent the pivotal joint. To this end, many braces include one or more cuff members situated around limb structure sites above and below the supported joint. As is apparent, the cuff members are responsible for stabilizing the support member and therefore must be well secured to their associated limbs.

To accomplish such securement, many conventional cuff members are typically provided with one or more straps that are tightened around each limb structure and retained by buckles, hook-and-loop connections, or the like. Concurrently, the support member adjacent the joint may well be pivotal in a single plane and thus limit the normal multi-planar mobility of the joint. Such conventional construction is generally deficient, however, in that such cuff tightening is limited by strength of the user as well as flexibility of the cuff structure itself, while such joint construction prevents normal twistability between the pivoting limbs.

Other conventional cuff members have attempted to address this deficiency by incorporating a cuff tightening member along an inner surface of the cuff. Along these lines, the cuff tightening member can tighten the cuff around the user's limb by “pulling” the cuff around the limb. In this regard, cuff tightening member is tensioned until the cuff member is suitably adjusted. A common deficiency associated with the present-day cuff tightening member is that the cuff tightening member is typically required to be formed from a strong, durable metal material in order to pull/adjust the cuff member to the desired position. This material requirement typically increases the overall cost of the brace.

Therefore, there is a need is present for an exteriorly positionable anatomical brace wherein the tightness of the cuff members can be easily adjusted via a cost-effective adjustment mechanism for effective brace engagement.

### BRIEF SUMMARY OF THE INVENTION

The present invention specifically addresses and alleviates the above-identified deficiencies in the art. There is provided a knee brace configured to be easily adjustable to conform to the size of the wearer's limb. Along these lines, the knee brace includes at least one adjustable cuff and an adjustment member coupled to the adjustable cuff for effectuating adjustment of the cuff. The adjustment member is generally exteriorly positioned relative to the cuff to allow the adjustment member to apply a compressing force upon the cuff to tighten the cuff on the wearer's limb. The adjustment member may be fabricated from a relatively low-cost material, such as a plastic, while still being capable of applying a suitable compressive force on the cuff.

According to one embodiment, there is provided an exteriorly positionable anatomical brace for stabilizing a uniting pivoting joint disposed between a first limb structure and a second limb structure of a living being. The brace includes an arcuate first cuff partially encompassable about the first limb structure, with the first cuff having an inner surface and a generally opposing outer surface. The first cuff is positionable relative to the first limb structure such that the inner surface faces toward the first limb structure and the outer surface faces away from the first limb structure. The first cuff defines an adjustable curvature.

The brace additionally includes an arcuate second cuff partially encompassable about the second limb structure, wherein the second cuff includes an inner surface and a generally opposing outer surface. The second cuff is positionable relative to the second limb structure such that the inner surface faces toward the second limb structure and the outer surface faces away from the second limb structure. The second cuff defines an adjustable curvature.

An arcuate first adjustment member is positioned adjacent the outer surface of the first cuff and defines an adjustable curvature. The first adjustment member is coupled to the first cuff such that adjustment of the curvature of the first adjustment member causes adjustment of the curvature of the first cuff. An arcuate second adjustment member is positioned adjacent the outer surface of the second cuff and defining an adjustable curvature. The second adjustment member is coupled to the second cuff such that adjustment of the curvature of the second adjustment member causes adjustment of the curvature of the second cuff.

The inner surfaces of the first and second cuffs may define a concave configuration, and the outer surfaces of the first and second cuffs may define a convex configuration.

The first adjustment member may include a strip of rigid material having a first end portion and a second end portion, wherein the first end portion is fixedly connected to the first cuff and the second end portion is moveable relative to the first cuff. A first locking member may be connected to the first adjustment member and the first cuff. The first locking member may be configured to fix the position of the second end portion of the first adjustment member relative to the first cuff.

The brace may additionally include a first retention member coupled to the first cuff, the first retention member and the outer surface of the first cuff collectively defining a retention channel within which at least a portion of the first adjustment member resides. The first retention member may be integrally formed with the first cuff.

The brace may further include first and second brackets coupled to and extending from the outer surface of the first cuff in spaced relation to each other. The first adjustment member may be threaded through the first and second brackets to couple the first adjustment member to the first cuff.

The first adjustment member may include an inner surface and an outer surface, and may be coupled to the first cuff such that the inner surface of the first adjustment member faces the outer surface of the first cuff and the outer surface of the first adjustment member faces away from the outer surface of the first cuff.

The brace may additionally comprise at least one stiffener element coupled to the first adjustment member and configured to mitigate deformation of the first adjustment member as the curvature of the first adjustment member is adjusted. The stiffener element may include a slot through which the first adjustment member is disposed.

The present invention is best understood by reference to the following detailed description when read in conjunction with the accompanying drawings.

### BRIEF DESCRIPTION OF THE DRAWINGS

These as well as other features of the present invention will become more apparent upon reference to the drawings wherein:

FIG. 1 is an upper perspective view of a knee brace shown on a user depicted in phantom, the knee brace including a first cuff and a first adjustment member for adjusting the curvature of the first cuff, as well as a second cuff and a second adjustment member for adjusting the curvature of the second cuff;

FIG. 2 is an upper perspective view of the first cuff and first adjustment member, wherein a user is employing an unlocking tool to release a locking cap for adjustment of the first adjustment member;

FIG. 3 is a partial upper perspective view of the first cuff, first adjustment member and locking cap shown in FIG. 2, with the locking cap being disengaged with the first adjustment member and the first cuff to allow for selective adjustment of the first adjustment member relative to the first cuff;

FIG. 4 is a top view of the first cuff and strap encompassing a first limb structure;

FIG. 5 is a top view of the second cuff and strap encompassing a second limb structure;

FIG. 6 is an upper perspective view of the first cuff;

FIG. 7 is a partial upper perspective view of the brace having a stiffening element coupled to the first adjustment member; and

FIG. 8 is a top view of the partial brace shown in FIG. 7, showing the stiffening element coupled to the first adjustment member.

Common reference numerals are used throughout the drawings and detailed description to indicate like elements.

### DETAILED DESCRIPTION OF THE INVENTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

Referring now to the drawings, wherein the showings are for purposes of illustrating a preferred embodiment of the present invention and not for purposes of limiting the same, there is shown a knee brace 10 designed to allow for selective adjustment of the size of the brace 10 to conform to the size of the user's leg. More specifically, the knee brace 10 includes flexible upper and lower cuffs 12, 14 along with corresponding upper and lower adjustment members 16, 18 for adjusting the curvature of the upper and lower cuffs 12, 14 to conform to the curvature/size of the user's leg. The upper and lower adjustment members 16, 18 are disposed adjacent an outer surface of the upper and lower cuffs 12, 14, which allows the upper and lower adjustment members 12, 14 to compress the upper and lower cuffs 12, 14 toward the user's leg to fit the cuff members 12, 14 to the size of the user.

A significant distinction between the adjustment members 16, 18 of the present invention and conventional tensioning members used with previous cuff members is the external position of the present adjustment members 16, 18. Whereas the conventional tensioning members were disposed adjacent an inner surface of the previous cuff members, the present adjustment members 16, 18 are positioned external to the respective cuffs 12, 14, which allows the adjustment members 16, 18 to compress or push the cuff 12, 14 to tighten the cuffs 12, 14 to the user's limb. In contrast, conventional tensioning members pulled the cuff members toward the user's limb, which required that the tensioning member was constructed from a stronger, more durable material. The external placement of the adjustment members 16, 18 allows the adjustment members 16, 18 to be constructed from a more cost-effective material, which lowers the overall cost of the brace 10.

The term “curvature” is used herein in relation to the cuffs 12, 14, as well as in relation to the user's limb structure, to characterize the arcuate nature of the cuffs 12, 14 or limb structure. Generally speaking, the curvature of a straight line is equal to zero, whereas the curvature of a circle is some quantity greater than zero. Thus, if the radius of the circle is large, then the curvature is small, and conversely, if the radius of the circle is small, then the curvature is large. Therefore, the curvature of a circle may be defined as the reciprocal of the radius. In the case of the cuffs 12, 14, the curvature is decreased by increasing the radius defined by the cuffs 12, 14 (i.e., making the cuffs 12, 14 more flat), while the curvature is increased by decreasing the radius defined by the cuffs 12, 14 (i.e., making the cuffs 12, 14 less flat).

Those skilled in the art will readily appreciate that the knee brace 10 depicted in the Figures is one exemplary embodiment of an exteriorly positionable anatomical brace 10 designed to stabilize a uniting pivoting joint disposed between a first limb structure and a second limb structure of a living being. In the case of a knee brace 10, the first and second limb structures include the thigh and shin/calf disposed on opposite side of a user's knee. However, it is understood that the scope of the present invention is not limited to a knee brace 10, and that braces for other pivoting joints, such as an elbow, may also fall within the scope of the present invention.

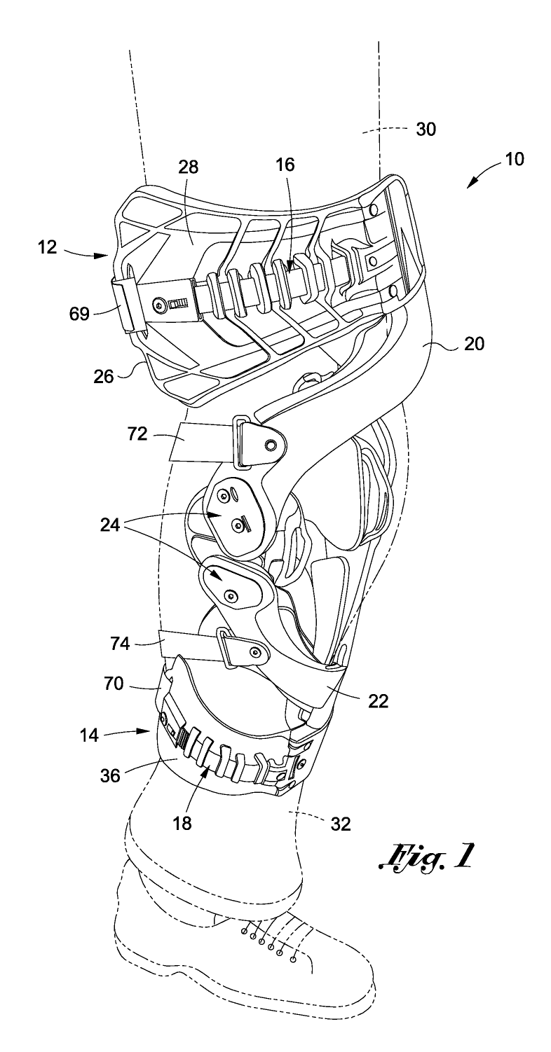

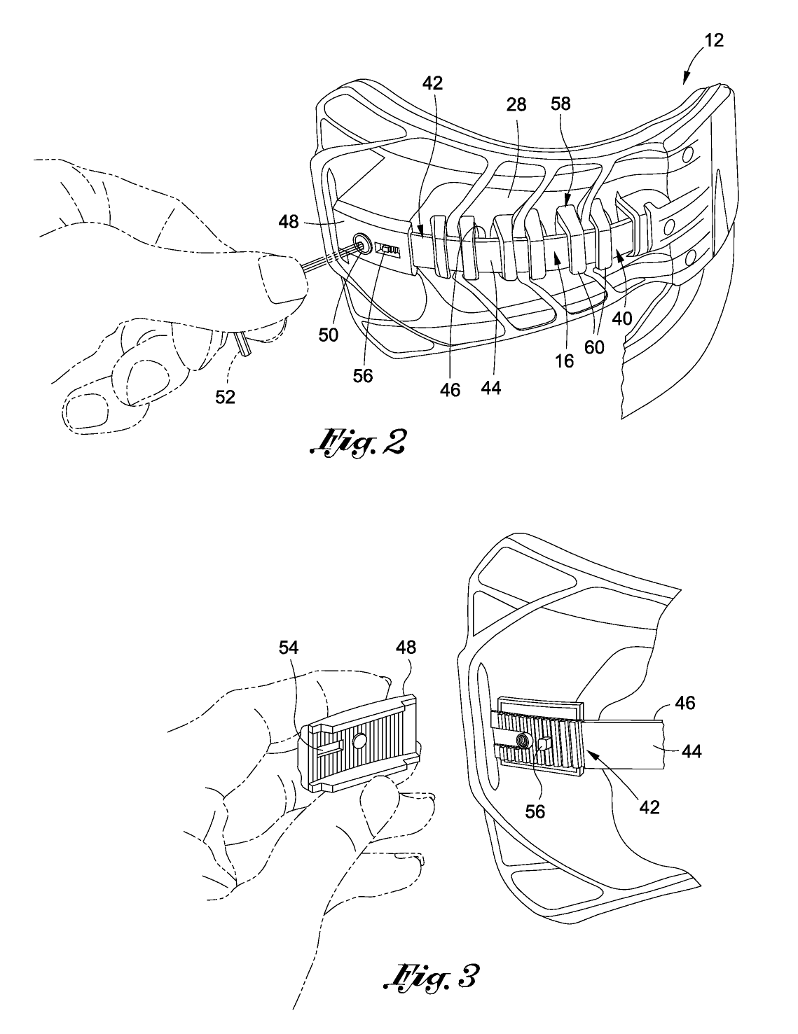

The knee brace 10 shown in FIG. 1 includes an upper cuff 12 (i.e., a first cuff) coupled to a rigid upper frame member 20, and a lower cuff 14 (i.e., a second cuff) coupled to a rigid lower frame member 22. The upper and lower frame members 20, 22 are pivotally coupled to each other via a pair of pivoting joint elements 24 disposed on opposed sides of the knee (i.e., one pivoting joint element 24 is disposed on the medial side of the knee and the other pivotal joint element is disposed on the lateral side of the knee). For a more detailed discussion of an exemplary pivoting joint element 24, please refer to U.S. Pat. No. 6,464,657 entitled Anatomical Joint Brace, owned by Asterisk.Asterisk, LLC, the owner of the present patent application, the contents of U.S. Pat. No. 6,464,657 being expressly incorporated herein by reference.

The upper frame member 20 extends from a pivoting joint element 24 upwardly along the user's leg to the user's thigh, while the lower frame member 22 extends from a pivoting joint element 24 down the user's leg to the user's shin/calf. The user's leg is depicted in phantom to provide a frame of reference as to the position of the brace 10 when placed on the user's leg. The frame members 20, 22 in combination with the pivoting joint elements 24 limit the pivotal range of motion of the user's leg to protect the user's knee from injury.

The first cuff 12 is an arcuate member which is partially encompassable about the first limb structure 30, i.e., the user's thigh depicted in FIG. 1. The first cuff 12 includes an inner surface 26 (see FIG. 4) and a generally opposing outer surface 28 (see FIGS. 1 and 4). The first cuff 12 is positionable relative to the first limb structure 30 such that the inner surface 26 of the first cuff 12 faces toward the first limb structure 30 and the outer surface 28 faces away from the first limb structure 30. In this regard, the inner surface 26 of the first cuff 12 defines a generally concave configuration which partially encompasses or extends about a portion of the first limb structure 30. The outer surface 28 of the first cuff 12 defines a generally convex configuration and faces away from the first limb structure 30 when the brace 10 is worn by the user.

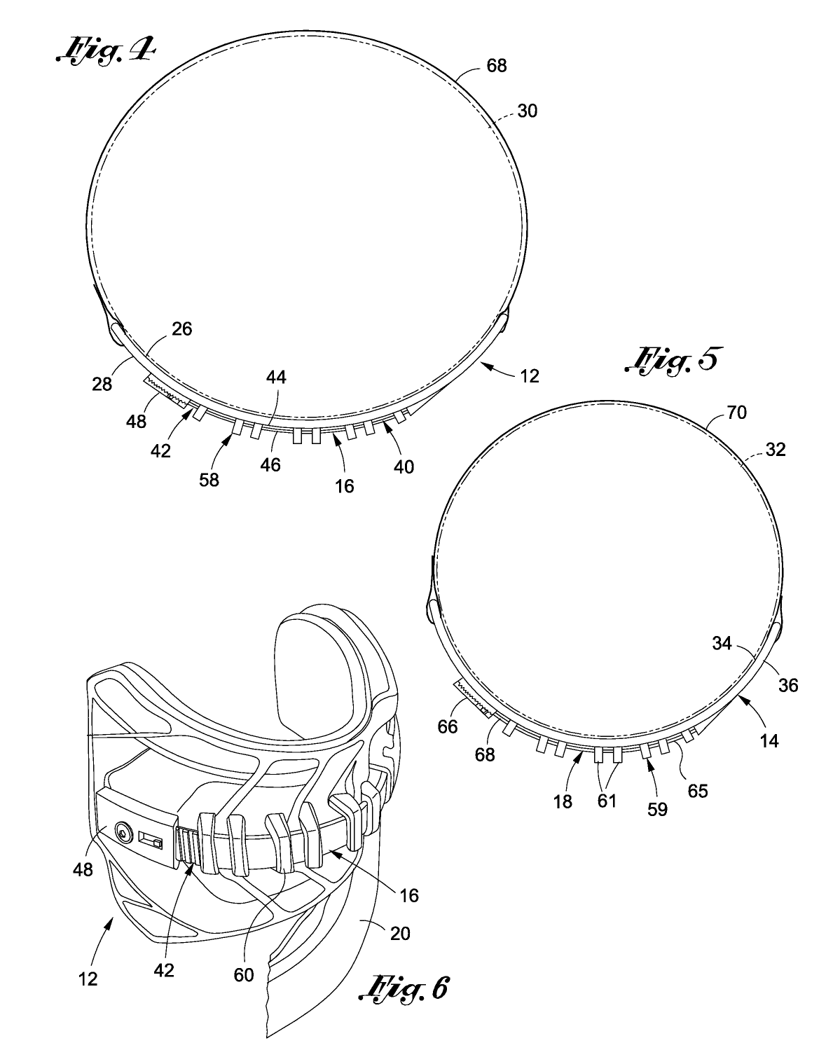

The second cuff 14 is similar to the first cuff 12 in that it defines an arcuate configuration; however, according to various aspects of the present invention, the size of the second cuff 14 is different from the first cuff 14 in view of the fact that the first cuff 12 is designed to partially encompass the first limb structure 30, whereas the second cuff 14 is designed to partially encompass the second limb structure 32, which is typically different in size compared to the first limb structure 30. The second cuff 14 includes an inner surface 34 (see FIG. 5) and a generally opposing outer surface 36 (see FIGS. 1 and 5). The second cuff 14 is positionable relative to the second limb structure 32 such that the inner surface 34 faces toward the second limb structure 322 and the outer surface 36 faces away from the second limb structure 32. The inner surface 34 defines a generally concave configuration, whereas the outer surface 36 defines a generally convex configuration.

Various aspects of the present invention are related to the size adjustment of the first and second cuffs 12, 14 to adapt the cuffs 12, 14 to fit properly on the wearer. Along these lines, one embodiment of the knee brace 10 includes an arcuate first adjustment member 116 coupled to the first cuff 12 for adjusting the curvature of the first cuff 12. A unique aspect of various embodiments of the brace 10 is the external position of the first adjustment member 116 relative to the first cuff 12. In the exemplary embodiment, the first adjustment member 16 is positioned adjacent the outer surface 28 of the first cuff 12. This is in contrast to many existing braces which include cuff adjustment mechanisms disposed adjacent the inner surface of the cuff. The external position of the first adjustment member 16 relative to the first cuff 12 allows the first adjustment member 16 to compress or constrict around the first cuff 12 to increase the curvature of the first cuff 12 (i.e., make the first cuff 12 less flat).

The first adjustment member 12 defines an adjustable curvature and is coupled to the first cuff 12 such that adjustment of the curvature of the first adjustment member 12 causes adjustment of the curvature of the first cuff 12. In the exemplary embodiment, the first adjustment member 16 includes a strip of rigid, preferably plastic, material having a first end portion 40 (see FIGS. 2 and 4), a second end portion 42 (see FIGS. 2 and 4), as well as opposing inner and outer surfaces 44, 46 (see FIGS. 2 and 4). The first end portion 40 is fixedly connected to the first cuff 12 and the second end portion 42 is moveable relative to the first cuff 12. In the exemplary embodiment, the first end portion 40 is captured within a channel formed within the first cuff 12 and is then riveted to the first cuff 12 to secure the first end portion 40 to the first cuff 12. The first adjustment member 16 is coupled to the first cuff 12 such that the inner surface 44 of the first adjustment member 16 faces at least a portion of the outer surface 28 of the first cuff 12 and the outer surface 46 of the first adjustment member 16 faces away from the outer surface 28 of the first cuff 12.

The second end portion 42 of the first adjustment member 16 is coupled to the first cuff 12 via a first locking member 48 (see FIGS. 2 and 4). The first locking member 48 and the second end portion 42 of the first adjustment member 16 are configured to allow for selective positional adjustment of the second end portion 42 relative to the first cuff 12. In the exemplary embodiment, the first locking member 48 and the second end portion 42 define complimentary engagement surfaces which facilitate the selective positional adjustment of the second end portion 42 relative to the first cuff 12. The first locking member 48 is secured to the first cuff 12 via a screw 50 using a locking tool 52, such as a hex key, screw driver or the like. When the first locking member 48 is secured to the first cuff 12, the position of the second end portion 42 is fixed relative to the first cuff 12. Therefore, to adjust the position of the second end portion 42 relative to the first cuff 12, which in turn adjusts the curvature of the first cuff 12, the first locking member 48 is loosened from the first cuff 12 and the second end portion 42 so as to allow selective positioning of the second end portion 42 relative to the first cuff 12. When the desired position of the second end portion 42 relative to the first cuff 12 is achieved, the first locking member 48 is engaged with the second end portion 42 and the first cuff 12 and the screw 50 is tightened to maintain the second end portion 42 in the desired position. The complimentary toothed surfaces of the first locking member 48 and the second end portion 42 mitigate movement of the second end portion 42 relative to the first locking member 48 when the first locking member 48 is engaged with the second end portion 42.

According to one embodiment, the first locking member 48 includes a window 54 and the second end portion 42 of the adjustment member 16 includes a position indicator tab 56 which extends through the window 54 to indicate the position of the second end portion 42 relative to the first locking member 48, as well as the first cuff 12. In this regard, the user may measure the curvature of the cuff via the position of the indicator tab 56 within the window 54.

Although the exemplary embodiment shows a first locking member 48 that is secured to the first adjustment member 16 via a screw, such that adjustment of the first adjustment member 16 requires loosening of the screw, it is contemplated that other embodiments of the first locking member 48 may be used without departing from the spirit and scope of the present invention. Along these lines, other embodiments of the locking member 48 may include binding-type locking members or other types of locking members which allow for selective adjustment of the adjustment member 16.

The brace 10 further includes a first retention member 58 coupled to the first cuff 12. The first retention member 58 and the outer surface 28 of the first cuff 12 collectively define a retention channel within which at least a portion of the first adjustment member resides. According to one embodiment, the first retention member 58 is integrally formed with the first cuff 12. In the exemplary embodiment, the first retention member 58 includes several brackets 60 which are coupled to, and extend from the outer surface 28 of the first cuff 12 in spaced relation to each other. The first adjustment member 16 is threaded through the brackets 60 to couple the first adjustment member 16 to the first cuff 12. When the curvature of the first adjustment member 16 is increased, the brackets 60 maintain the first adjustment member 16 is close proximity to the first cuff 12.

The second cuff 14 is configured similar to the first cuff 12 described above, in that the second cuff 14 is configured to have an arcuate adjustment member 18 generally exteriorly positioned relative thereto for adjusting the degree of curvature of the second cuff 14. The configuration of the second adjustment member 18 is similar to the first engagement member 16, and the engagement between the second engagement member 18 and the second cuff 14 is similar to the first engagement member 16 and the first cuff 12. The second adjustment member 18 includes a first end portion 65 fixedly coupled to the second cuff 14, as well as a second end portion 68 moveably coupled to the second cuff 14 for adjusting the curvature of the second cuff 14. The second adjustment member 18 is positioned adjacent the outer surface 36 of the second cuff 14 such that the external position of the second adjustment member 18 relative to the second cuff 14 allows the second adjustment member 18 to compress or constrict around the second cuff 114 to increase the curvature of the second cuff 14 (i.e., make the second cuff 14 less flat).

The second cuff 14 additionally includes a second retention member 59 (see FIG. 5) coupled to the second cuff 14. The second retention member 59 and the outer surface 36 of the second cuff 14 defines a retention channel within which at least a portion of the second adjustment member 18 resides. According to one embodiment, the second retention member 59 is integrally formed with the second cuff 14 and includes a plurality of brackets 61, similar to the brackets 60 discussed above in relation to the first cuff 12. The second adjustment member 18 is threaded through the brackets 61 to couple the second adjustment member 18 to the second cuff 14.

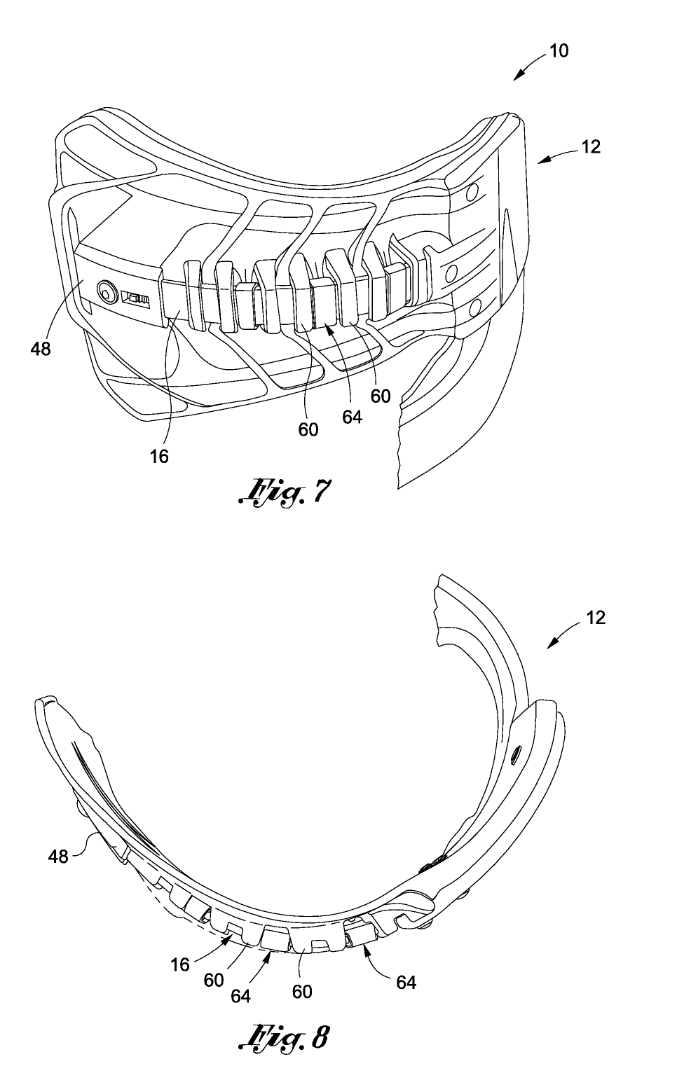

Referring now specifically to FIGS. 7 and 8, one embodiment of the knee brace 10 includes at least one stiffener element 64 coupled to the first adjustment member 16 or second adjustment member 18, wherein the stiffener element 64 is configured to mitigate deformation of the respective one of the first and second adjustment member 16, 18 as the curvature of the first and second adjustment members 16, 18 is adjusted. The stiffener element 64 defines a slot through which the adjustment member 16, 18 is disposed.

With the primary structural features of the brace 10 being described above, the following discussion focuses on operation of the brace 10. The user dons the brace 10 by placing the brace 10 adjacent the pivoting joint such that the first cuff 12 is positioned adjacent the first limb member 30 while the second cuff 14 is positioned adjacent the second limb member 32. The curvature of the first cuff 12 is adjusted to conform to the size of the first limb structure 30. The curvature of the first cuff 12 is adjusted by loosening the first locking member 48 and sliding the moveable end portion 42 of the first adjustment member 16 relative to the first cuff 12. The sliding motion of the moveable end portion 42 modifies the curvature of the first adjustment member 16, which in turn, modifies the curvature of the first cuff 12. When the curvature of the first cuff 12 is appropriately sized to conform to the first limb structure 30, the first locking member 48 is secured to maintain the position of the first adjustment member 16, as well as the curvature of the first cuff 12.

The user also makes a similar adjustment to the second cuff 14 to adjust the size of the second cuff 14 to the second limb structure 32. The second cuff 14 is adjusted by loosening a second locking member 66 to allow for slidable adjustment of the moveable end portion 68 of the second adjustment member 18, which in turn adjusts the curvature of the second cuff 14. When the desired curvature is achieved, the second locking member 66 is tightened to lock the second adjustment member 18 to maintain the desired curvature of the second cuff 14.

With the first and second cuffs 12, 14 properly adjusted, the user may secure the brace 10 to the user. In this regard, the brace 10 may include a series of straps and fasteners used to secure the brace 10 to the user's limb. In the exemplary embodiment, the brace 10 includes an upper cuff strap 69, a lower cuff strap 70, an upper frame strap 72 and a lower frame strap 74. The straps 69, 70, 72, 74 may include hook and loop fasteners, buttons, snaps or other mechanical fasteners known in the art for securing the straps 69, 70, 72, 74 to the user's limb. Furthermore, although the exemplary embodiment includes four straps 69, 70, 72, 74, it is understood that any number of straps may be employed without departing from the spirit and scope of the present invention.

Additional modifications and improvements of the present invention may also be apparent to those of ordinary skill in the art. Thus, the particular combination of components and steps described and illustrated herein is intended to represent only certain embodiments of the present invention, and is not intended to serve as limitations of alternative devices and methods within the spirit and scope of the invention.

What is claimed is:

1. An exteriorly positionable anatomical brace for stabilizing a uniting pivoting joint disposed between a first limb structure and a second limb structure of a living being, the brace comprising:
an arcuate first cuff partially encompassable about the first limb structure, the first cuff having an inner surface and a generally opposing outer surface, the first cuff being positionable relative to the first limb structure such that the inner surface faces toward the first limb structure and the outer surface faces away from the first limb structure, the first cuff defining an adjustable curvature;
an arcuate second cuff partially encompassable about the second limb structure, the second cuff having an inner surface and a generally opposing outer surface, the second cuff being positionable relative to the second limb structure such that the inner surface faces toward the second limb structure and the outer surface faces away from the second limb structure, the second cuff defining an adjustable curvature;
an arcuate first adjustment member positioned adjacent the outer surface of the first cuff, the first adjustment member defining an adjustable curvature, the first adjustment member being coupled to the first cuff such that adjustment of the curvature of the first adjustment member causes adjustment of the curvature of the first cuff; and
an arcuate second adjustment member positioned adjacent the outer surface of the second cuff, the second adjustment member defining an adjustable curvature, the second adjustment member being coupled to the second cuff such that adjustment of the curvature of the second adjustment member causes adjustment of the curvature of the second cuff.

2. The exteriorly positionable anatomical brace as recited in claim 1 wherein the inner surfaces of the first and second cuffs define a concave configuration, and the outer surfaces of the first and second cuffs define a convex configuration.

3. The exteriorly positionable anatomical brace as recited in claim 1 wherein the first adjustment member includes a strip of rigid material having a first end portion and a second end portion, the first end portion being fixedly connected to the first cuff and the second end portion being moveable relative to the first cuff.

4. The exteriorly positionable anatomical brace as recited in claim 3 further comprising:
a first locking member connected to the first adjustment member and the first cuff, the first locking member being configured to fix the position of the second end portion of the first adjustment member relative to the first cuff.

5. The exteriorly positionable anatomical brace as recited in claim 1 further comprising:
a first retention member coupled to the first cuff, the first retention member and the outer surface of the first cuff collectively defining a retention channel within which at least a portion of the first adjustment member resides.

6. The exteriorly positionable anatomical brace as recited in claim 5 wherein the first retention member is integrally formed with the first cuff.

7. The exteriorly positionable anatomical brace as recited in claim 1 wherein further comprising first and second brackets coupled to and extending from the outer surface of the first cuff in spaced relation to each other, the first adjustment member being threaded through the first and second brackets to couple the first adjustment member to the first cuff.

8. The exteriorly positionable anatomical brace as recited in claim 1 wherein the first adjustment member including an inner surface and an outer surface, the first adjustment member being coupled to the first cuff such that the inner surface of the first adjustment member faces the outer surface of the first cuff and the outer surface of the first adjustment member faces away from the outer surface of the first cuff.

9. The exteriorly positionable anatomical brace as recited in claim 1, further comprising at least one stiffener element coupled to the first adjustment member and configured to mitigate deformation of the first adjustment member as the curvature of the first adjustment member is adjusted.

10. The exteriorly positionable anatomical brace as recited in claim 9, wherein the at least one stiffener element includes a slot through which the first adjustment member is disposed.

11. The exteriorly positionable anatomical brace as recited in claim 1, wherein at least one of the first and second adjustment members are formed from a plastic material.

12. An exteriorly positionable anatomical brace for stabilizing a uniting pivoting joint disposed between a first limb structure and a second limb structure of a living being, the brace comprising:
an adjustable arcuate first cuff partially encompassable about the first limb structure, the first cuff having an inner surface and a generally opposing outer surface, the first cuff being positionable relative to the first limb structure such that the inner surface faces toward the first limb structure and the outer surface faces away from the first limb structure;
an adjustable arcuate second cuff partially encompassable about the second limb structure, the second cuff having an inner surface and a generally opposing outer surface, the second cuff being positionable relative to the second limb structure such that the inner surface faces toward the second limb structure and the outer surface faces away from the second limb structure;
an arcuate first adjustment member positioned adjacent the outer surface of the first cuff; and
an arcuate second adjustment member positioned adjacent the outer surface of the second cuff;
the first cuff and first adjustment member being configured such that at least a portion of the first adjustment member is compressed upon the first adjustment member to conform the first cuff to the first limb structure;
the second cuff and second adjustment member being configured such that at least a portion of the second adjustment member is compressed upon the second adjustment member to conform the second cuff to the second limb structure.

13. The exteriorly positionable anatomical brace as recited in claim 12 wherein the first adjustment member includes a strip of rigid material having a first end portion and a second end portion, the first end portion being fixedly connected to the first cuff and the second end portion being moveable relative to the first cuff.

14. The exteriorly positionable anatomical brace as recited in claim 13 further comprising:
a first locking member connected to the first adjustment member and the first cuff, the first locking member being configured to fix the position of the second end portion of the first adjustment member relative to the first cuff.

15. The exteriorly positionable anatomical brace as recited in claim 12 further comprising:
a first retention member coupled to the first cuff, the first retention member and the outer surface of the first cuff collectively defining a retention channel within which at least a portion of the first adjustment member resides.

16. The exteriorly positionable anatomical brace as recited in claim 15 wherein the first retention member is integrally formed with the first cuff.

17. The exteriorly positionable anatomical brace as recited in claim 12 wherein the first adjustment member including an inner surface and an outer surface, the first adjustment member being coupled to the first cuff such that the inner surface of the first adjustment member faces the outer surface of the first cuff and the outer surface of the first adjustment member faces away from the outer surface of the first cuff.

18. The exteriorly positionable anatomical brace as recited in claim 12, further comprising at least one stiffener element coupled to the first adjustment member and configured to mitigate deformation of the first adjustment member as the curvature of the first adjustment member is adjusted.

19. The exteriorly positionable anatomical brace as recited in claim 12, wherein at least one of the first and second adjustment members are formed from a plastic material.

20. An exteriorly positionable anatomical brace for stabilizing a uniting pivoting joint disposed between a first limb structure and a second limb structure of a living being, the brace comprising:
an adjustable arcuate first cuff partially encompassable about the first limb structure, the first cuff having an inner surface and a generally opposing outer surface, the first cuff being positionable relative to the first limb structure such that the inner surface faces toward the first limb structure and the outer surface faces away from the first limb structure; and
an arcuate first adjustment member positioned adjacent the outer surface of the first cuff;
the first cuff and first adjustment member being configured such that at least a portion of the first adjustment member is compressed upon the first adjustment member to conform the first cuff to the first limb structure.
